SEQUENCE LISTING (2 sequences)

SEQ ID NO: 1 - DNA, 10 bp, Artificial Sequence
  Synthetic probe
  misc_feature (4)..(7): n is a, c, g, or t
actnnnncta

SEQ ID NO: 2 - DNA, 12 bp, Artificial Sequence
  Synthetic probe
  misc_feature (3)..(5): n is a, c, g, or t
  misc_feature (8)..(10): n is a, c, g, or t
acnnntgnnnac

(19) United States
(12) Patent Application Publication
(10) Pub. No.: US 2018/0051331 A1
(43) Pub. Date: Feb. 22, 2018

(54) Methods for Mapping Bar-Coded Molecules for Structural Variation Detection and Sequencing

(71) Applicant: Singular Bio, Inc., San Francisco, CA (US)

(72) Inventor: Hywel Bowden Jones, San Francisco, CA (US)

(73) Assignee: Singular Bio, Inc., San Francisco, CA (US)

(21) Appl. No.: 15/581,971

(22) Filed: Apr. 28, 2017

Related U.S. Application Data
(63) Continuation of application No. 14/373,113, filed on Jul. 18, 2014, now abandoned.
(60) Provisional application No. 61/587,861, filed on Jan. 18, 2012.

(51) Int. Cl. C12Q 1/68; G06F 19/20

(52) U.S. Cl. CPC C12Q 1/6869; C12Q 2563/185; G06F 19/20

(57) ABSTRACT

The invention includes methods for optimally designing probes and analyzing data from sequence-byhybridization and related methods of stretched molecules or other experimental approaches that provide local information. An exemplary method of analyzing a nucleic acid sample may comprise: selecting a group of one or more labeled oligonucleotide probe(s), contacting at least one of the group of the labeled oligonucleotide probe(s) to at least one nucleic acid molecule(s) from the nucleic acid sample, wherein the nucleic acid molecule(s) is stretched, and correlating one or more point(s) of contact to a structural characteristic of the nucleic acid sample. In some embodiments, the nucleic acid molecule(s) is deoxyribonucleic acid (DNA) and/or the method of contacting is hybridization or ligation.

A computer readable text file, entitled “SequenceListing.txt,” created on or about Apr. 28, 2017 with a file size of about 1 kb contains the sequence listing for this application and is hereby incorporated by reference in its entirety.

### FIELD OF THE INVENTION

The invention includes methods for optimally designing probes and analyzing data from sequence-by-hybridization and related methods on stretched molecules or other experimental approaches that provide local information.

### BACKGROUND TO THE INVENTION

Individual molecules may be bar-coded in a variety of ways. In one approach, short fluorescently labeled oligonucleotide probes are hybridized to the molecule. The molecule is stretched out on a surface either before, during or after the hybridization. It is then imaged to identify the points of hybridization along its length. A labeled molecule appears as a row of points of light and the distance between them represent a measure of the physical distance between occurrences of the probe's target sequence on the molecule.

In an idealized version, many molecules are stretched or linearized and imagined simultaneously by packing them at high density on a surface.

Probes of various designs may be used including, but not limited to, probes of varying length. For example, the probes may vary from 1 basepair (bp) to hundreds of bp's in length. The probes may be DNA or RNA or protein or a combination thereof. The probes may target any nucleic acid including DNA or RNA. The probes may be UV sensitive to allow cross linking. The probe may be a Peptide Nucleic Acids (PNA), gammaPNA, Locked Nucleic Acids (LNA) or other type of oligos. Probes may contain degenerative nucleotides, universal bases or other gaps or spacers (for example, a probe could be ACTNNNNCTA, where the N will hybridize to any nucleotide). Probes may be labeled using fluorescent dyes of specified wavelength (e.g. quantum dots). Probes may be labeled with tags of specific weight and may be labeled before or after the hybridization. Probes may be labeled with tags of specific structure and may be labeled before or after the hybridization. They may include elements that quench the dye and may target single-stranded (ss) or double-stranded (ds) molecules. There may be one or more enzymatic steps in attaching the probe to the molecule, and/or one or more biochemical steps in attaching the probe to the molecule. The assay described herein may occur in solution or after the molecules are stretched on a surface. The probes may be removable after imaging and/or quenched after imaging. Probes may be used in sequential or parallel manner.

The target molecule may have a variety of properties including, but not limited to, being DNA or RNA or protein or a combination of these, being genomic, mitochondrial, viral, bacterial, human, non-human, synthetic or other kinds of sequence, being single-stranded (ss) or double-stranded (ds) molecules, being of any length from 1 bp to 100,000,000,000 bp's. Ideally, they will be at least 5,000 bp's in length, or being composed of a contiguous sequence or chimeric and composed of sub-units.

Stretching or linearizing or measuring may occur on a variety of ways including, but not limited to, on a solid substrate such as a glass slide, on an etched surface, in a channel, micro-channel or nano-channel or other fabricated device, through a nanopore, and/or on a treated surface (e.g. a surface functionalized with capture oligos targeted at specific molecules).

The process of stretching or linearizing or measuring may have other properties including, but not limited to, one or more molecules being aligned spatially, deposited at different times, stretched of linearized simultaneously, stretched or linearized at any density on a surface, and/or having certain characteristics (for example, being longer than a minimum length).

Stretching may occur in a variety of ways including, but not limited to, via liquid flow which pulls the molecules in a given direction, gaseous flow which pulls the molecules in a given direction, evaporation where the receding water droplet stretches the molecules, dipping into a liquid, where the process of withdrawal stretches the molecules, a physical stretching, where a solid is dragged over the surface to stretch the molecules, passing through a nanopore, and/or passing through a channel, micro-channel or nano-channel or other fabricated device.

Imaging may occur in a variety of ways including, but not limited to, light-based imaging using a microscope or similar device, electronic detection using a nanopore, imaging may occur when the probes are stationary, imaging may occur when the probes are in motion (e.g., in a liquid flow), and/or imaging may occur in a continuous or step-by-step manner.

### SUMMARY OF THE INVENTION

The invention relates to a method of analyzing a nucleic acid sample, comprising: selecting a group of one or more labeled oligonucleotide probe(s), contacting at least one of the group of the labeled oligonucleotide probe(s) to at least one nucleic acid molecule(s) from the nucleic acid sample, wherein the nucleic acid molecule(s) is stretched, and correlating one or more point(s) of contact to a structural characteristic of the nucleic acid sample. In some embodiments, the nucleic acid molecule(s) is deoxyribonucleic acid (DNA) and/or the method of contacting is hybridization or ligation. The method described herein may further include: imaging points of contact along the nucleic acid molecules and measuring the distance between the nucleic acid molecules and/or sequencing at least one part of the nucleic acid molecule(s). Such sequencing may be performed by using information on the points of contact and the distance between the nucleic acid molecules. In some embodiments, the labeled oligonucleotide probe(s) are selected from a group of 4096 possible oligonucleotide probes having at least 6 nucleotides or consists of the group of 4096 possible oligonucleotide probes. In some embodiments, the nucleic acid molecule(s) described herein is a whole genome sequence.

In additional embodiments, the method described herein may further comprise detecting an error(s) in either the location of the contacting or the distance between contact points, quantifying the error(s), and/or correcting the error(s). In further embodiments, the method described herein may further comprise sequencing the nucleic acid molecule(s), reconstructing a nucleic acid sequence from the labeled oligonucleotide probe(s) that have not been contacted to the nucleic acid molecule(s), comparing the sequenced nucleic acid molecule(s) and the reconstructed nucleic acid sequence, and using this information in correcting an error(s).

In one aspect, the nucleic acid sample may comprise either single or double stranded nucleic acid molecule(s), or a combination thereof. In some embodiments, the nucleic acid sample comprises double stranded nucleic acid molecules, and each step of the method is performed independently on each strand of nucleic acid molecule.

In another aspect, the labeled oligonucleotide probe(s) described herein may comprise a spacer. For example, the labeled oligonucleotide probe(s) may comprise a spacer that is located to optimize reconstruction of genomic information. In some embodiments, the labeled oligonucleotide probe(s) comprises a spacer and/or a degenerative nucleotide, and the labeled oligonucleotide probe(s) comprises 6 or fewer non-spacer nucleotides.

In another aspect, the labeled oligonucleotide probe(s) is less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7 or 6 nucleotide long.

In another aspect, the nucleic acid molecule is stretched before or after the contacting with the labeled oligonucleotide probe(s). In some embodiments, the nucleic acid molecule(s) is not nicked by the labeled oligonucleotide probe(s).

### BRIEF DESCRIPTION OF THE DRAWINGS

FIG. 1 depicts the mapping of molecules either to a reference of to each other.

FIG. 2 depicts Five probe maps (each in a different color) are aligned (top) allowing the set of probes in specific 1000 bp intervals to be identified.

FIG. 3 depicts an assembly by tiling using the observed subset of timer probes.

FIG. 4 shows that an inversion is easy to detect as the bar-code pattern is inverted between the sample (top) and the reference (bottom).

FIG. 5A, FIG. 5B, and FIG. 5C shows examples of locating a molecule against the reference using custom algorithms based on the sum of the squares of the distances.

FIG. 6 shows relative accuracy for detecting a variant against the scenario with zero missing probes (shown on the left vertical axis) against the missing probe rate (x-axis) with 10% cross-hybridization. The trend line shows the average number of assemblies with equal or greater match than the correct assembly (enumerated on the right vertical axis).

FIG. 7 shows Relative accuracy for detecting a variant against the scenario with zero missing probes (shown on the left vertical axis) against the missing probe rate (x-axis) with 50% cross-hybridization. The trend line shows the average number of assemblies with equal or greater match than the correct assembly (enumerated on the right vertical axis).

FIG. 8 shows relative accuracy for detecting a variant (against the scenario with zero missing probes) against the missing probe rate (x-axis). Each line represents a different level of cross-hybridization.

FIG. 9 depicts the ability to accurately assemble sequences using the custom algorithms. % w/Ref uses the reference only for assembly. % w/Secondary uses secondary information (as described in the text) to aid assembly.

FIG. 10 depicts that smaller assembly windows allow generally yield a smaller subset of the total probe set. That is, fewer distinct probes are observed for smaller assembly windows. Methods for determining the ability to accurate assembly sequence with assembly windows of different sizes have been developed.

### DESCRIPTION OF THE INVENTION

The method described herein may allow the location of bar-coded molecules or fragments (henceforth encompassed by the term “molecules”) either to a reference or to each other. This facilitates the detection of structural variation (SV), which are important in many human diseases, for example, Downs Syndrome and for sequencing the whole-genome using sequencing-by-hybridization (SbH) and related methods.

### Optimization of Probe Sequences

Algorithms allow the optimal design of probes. Optimization may be for a single probe or for a set of probes. Optimization may occur on many parameters including, but not limited to, distance between occurrences of the probe sequences in the reference sequence, molecule to be mapped or other sequence, distribution of the distances between occurrences of the probe sequences in the reference sequence, molecule to be mapped or other sequence, length of the probes (e.g. all the probes are 6 bps in length), distribution of the lengths of the probes, number of specific nucleotides, universal nucleotides, degenerate nucleotides or other gaps or spacers in the probe or probes, Locations of universal nucleotides, degenerate nucleotides or other gaps or spacers in the probe or probes, Number of over-lapping or related probes, GC-content of the probe, specific motifs of the probe (e.g. ACAC), assay conditions (e.g. hybridization conditions) for the probe or probes, specificity (e.g. how well it detects the target sequence compared to other sequences) of the probe or probes, and/or cross-hybridization rate of the probe or probes.

In some embodiments, optimization may be specific to the context. For example, a different set of probes may be more optimal for human than for mouse.

### Image Analysis

Individual molecule identification may include some or all of the following steps: individual molecules are identified on the image, the image may contain many molecules, molecule may overlap and identification of these points of overlap reduces error and maximizes the amount of information that may be extracted, molecules may not lie entirely straight and methods for determining their length more precisely may be used, molecules may be unevenly stretched and experimental methods (for example, using a intercalating dye) may be used to determine the relative stretching along the molecule, molecules may be unevenly stretched and algorithmic methods may be used to determine the relative stretching along the molecule (for example, if the molecules are of known lengths, a transformation may be applied), and/or molecules may be fragmented or broken and algorithms may be used to identify these component pieces.

Methods for incorporating the inaccuracy of the measurement may be modeled. For example, the software code in Appendix 2 uses an error function that is distributed with mean of 0 and variance of 1000. Many other error functions have been explored and these enable the choice of optimal instrument and experimental design for any given application. For example, some applications may require mapping of short molecules and in this case, higher accuracy would usually be needed to map the molecule as there are, on average, fewer observations of hybridization events. The software tool may be used to aid in instrument choice, experimental design and understanding of the likely power and accuracy of any experiment.

### Estimating Distance for Individual Molecules

Determining the distance between two probes on a molecule may include some or all of the following steps: the probe locations are identified for a single molecule on the image and/or distance is measured between the probes. In measing the distance, for fluorescent labels, the physical distance is measured on the image (e.g. the number of pixels between the probe locations represented by points of light). For nanopores, the time between probes because in the ideal case, the molecule is moving at a steady rate through the nanopore, so the time between probes is a linear function of the distance between. If the speed varies, more complex functions are optimal. If stretching is non-linear, more complex functions are applied to estimate the distance between probes. For example, a molecule may stretch differently at the point of attachment to the surface. Similarly, a molecule may stretch less at the unattached terminus where less force is applied. Stretching functions may be linear, exponential or step functions (for example, is the nucleic acid is changing to the S phase for part of its length) or any other function. In the simplest cases, the result for a single molecule is a vector of distances between consecutive probe hybridization (where hybridization may mean any assay or method of attaching the probes to the molecule and is taken to mean all these possibility throughout this text) events arrayed allowed the molecule. For example, if probe hybridization events 1 through 5 occur in that order along the molecule a vector of 4 elements describes the distances between probe hybridization events 1 and 2, 2 and 3, 3 and 4 and 4 and 5. This may be extended to any number of probe hybridization events. The results may be arrayed as a vector.

Factors affecting the measurement of distance between to occurrences of the probe hybridization events on a molecule include, but are not limited by, the following examples. In some embodiments, the resolution of the instrument (for example, the microscope) may limit the distances that may accurately be measured. Incorporating this information into the algorithm to estimate distance may improve accuracy. The instrument (for example, the microscope) may introduce bias into the measurement of distance. For example, it may be better at measuring short distances than long distances. Incorporating this information into the algorithm to estimate distance may improve accuracy. The distribution of the light emitted by the label or dye used to identify hybridization events where the probe has hybridized to the target molecule. Incorporating this distribution into the algorithm to estimate distance may improve accuracy. The intensity of the light emitted by the label or dye used to identify hybridization events where the probe has hybridized to the target molecule. Incorporating this intensity into the algorithm to estimate distance may improve accuracy.

More complex distance estimates may be generated using various approaches including, but not limited to, using a matrix of all pairwise distances between all pairs of probe hybridization events, using the mean, median, mode or other average of a set of measurements of the distance between two probe hybridization events on a given molecule (for example, distance may be repeatedly measured by re-scanning the molecule), using the distribution of distance measurements between two probe hybridization events on a given molecule (for example, distance may be repeatedly measured by re-scanning the molecule), and/or using the weighted average of a set of measurements of the distance between two occurrences of the probe on a given molecule (for example, distance may be repeatedly measured by re-scanning the molecule)

### Error Detection and Uncertainty

Error or uncertainty may occur in a number of ways including, but not limited to, cross-hybridization, where the probe hybridizes to a related sequence that is not the target (for example, a sequence that matches some subset of the probe's sequence), cross-hybridization, where the probe hybridizes to a unrelated sequence that is not the target (for example, the probe randomly, semi-randomly or non-randomly binds to the target), failed hybridization, where the probe fails to hybridize to a correct target sequence and gives missing data, and the probe may fail completely (zero correct hybridization events) or partially (not all correct hybridization events occur), and/or contamination by unbound probes that give false positive signals, contamination by non-target nucleic acids which allow the probes to bind. Error or uncertainty may occur also because of the following reasons. The probe sequence may be unknown and so all possible locations must be tested. For example, if the probe is known to be 6 bp in length, but the exact 6 bp sequence in unknown, all possible 6 bp locations must be tested. Multiple probes may be use simultaneously and require de-convolution. Probes may be hybridization consecutively, with one probe being removed from the target molecule before the next is introduced. In this case, incomplete removal of the first probe may lead to errors when measuring subsequent probes. These errors may occur in the methods, and an example is encapsulated in the software code in Appendix 1 and 2. These may be used to design optimal experiments as well as to assess power and accuracy and to map molecules and assemble sequence.

### Molecule Mapping

Molecules may be mapped to a reference sequence (for example, the human genome reference sequence). In some embodiments, the reference sequence may be generated in the same manner as the molecules are interrogated or produced using entirely different methods. The reference may be any other molecule. In the simplest case, the vector of distances for a given molecule is compared to the complete vector of distances from the reference sequence. In the simplest case, a perfect match gives the location of the molecule in the reference sequence. Matching may be any algorithm that quantifies the goodness-of-fit, probability of a match or other metric that determines how similar the molecule is to the particular location on the reference. A match may be determined to by any threshold, measure, metric, bound or in any other way. A given molecule may match to none, one or many locations in the reference. Imperfect matching may be allowed, For example, if more than a predetermined subset of the distances match for a given location in the reference, the molecule may be determined to match that location in the reference. For example, if 6 of 8 distances match a given location, the molecule may be judged to map to that location in the reference.

Typically, there will be error in the estimation of distance and matching between the molecule and reference will not be perfect and more complex algorithms will be preferred. A normalization step may be necessary in order to compare the molecules either to each other or to the reference. For example, the first distance may be set to 1 and the other distances on the molecule measured relative to it. When comparing the fit to a specific position in the reference, the first distance on the reference for the given location may be set to 1 and other distances on the reference measured relative to it.

A simple algorithm looks at the sum of the squares of the difference in distance between a molecule and the reference. For example, if the molecule has a distance vector M={10,20,10,50} defining the distances between five consecutive probe hybridization events and the reference has distance vector {50,10,25,10,50} defining the distances between five consecutive positions where the probe should hybridize, then the sum of the squares of the difference in distances for the molecule mapping to the first (left) position of the reference is, (10−50)²+(20−10)²+(10−25)²+(50−10)²=3,525 and the sum of the squares of the difference in distances for the molecule mapping to the second (right) position of the reference is, (10−10)²+(20−25)²+(10−10)²+(50−50)²=25. As such, the match is much better to the second (right) position than the first (left) position in the reference for this particular molecule since a lower score represents better fit.

More complex algorithms may be applied that favor specific factors including, but not limited to, long distances, short distances, repeated distances, strings of probes with zero distances between them.

Every position in the reference may be tested for fit. For example, if the probe matches at 100 locations and the molecule to be mapped has 5 occurrences of the probe sequence, the molecule may be tested at position 1, position 2, and so forth to position 95 moving along the reference. The match to each of the positions could be tested and a best fit determined. Positions 96 through to position 100 could also be tested but have fewer occurrences of the probe's target sequence than there are on the molecule to be mapped. That could be because, for example, by the molecule to be mapped only partially overlapping the reference.

A subset of the positions in the reference may be tested. The subset of positions tested could be random, non-random or selected on any criteria

One example of a mapping algorithm that incorporates error in distances is as follows. Assume the first position on the molecule to mapped of the probe's target sequence matches a position for the same sequence on the reference (called the first reference position). Measure the distance between the first and second position on the molecule to be mapped of the probe's target sequence. Measure the distance the between the first reference position and some or all of the occurrences of the probe's target sequence on the reference and label (these are other reference positions). Identify the reference positions whose distance from the first reference position most closely matches the distance between the first position and second position on the molecule to be mapped using a predetermined algorithm to measure the fit. Define the best fit position on the reference as the second position on the reference. Measure the distance between the second and third position on the molecule to be mapped of the probe's target sequence. Now measure the distances between the second position on the reference and all other positions on the reference. Identify the reference positions whose distance from the second reference position most closely matches the distance between the second position and third position on the molecule to be mapped using a predetermined algorithm to measure the fit. Define the best fit position on the reference as the third position on the reference. Continue this iteration for some or all of the positions on the molecule to be mapped. In a further enhancement, positions in the reference may be limited to that they are only used once (so the same occurrence of the probe's target sequence cannot be deemed to be the best fit with multiple positions of the molecule to be mapped).

Similar algorithms may be applied to distance matrices, averages, weighted averages and other more complex measures of distance on a molecule or in the reference.

In typical cases, the molecule and the reference will be from different samples and may differ in their structure. This will be reflected in differing distance measurements. In some cases, they may differ so much, the molecule cannot be mapped to the reference with high confidence. In an extreme case, the molecule and reference may be from different sources (for example, different species) and the molecule cannot be mapped to the reference. This inability to map may of itself be important as it may highlight contamination, sample mixing, errors in sample labeling and many other uses.

Errors such as missing hybridization or cross-hybridization will introduce errors into the distance measurements. These may be handled in a number of different ways including, but not limited to, deleting or ignoring aberrant information, down-grading, penalizing or down-weighting aberrant information, upgrading or up-weighting information known to be of high quality, and/or re-measuring aberrant information.

An example is encapsulated in the software code in Appendix 2. This may be used to design optimal experiments as well as to assess power and accuracy and to map molecules.

### Algorithmic Efficiency

For large reference sequences, the number of comparisons between the distance vector in the molecule and the reference may be large.

A variety or ways of speeding up the processing may be used including, but not limited to, the following examples, including comparing the match from each location to the current best match location. For example, if the current best match using a sum of the squares of the difference in distances between the molecule and a specific location in the reference is 100, any location in the reference that has a partial sum of the squares of the difference in distances between the molecule and a particular location in the reference that is greater than 100 need not be fully evaluated. This relies on the fact that the sum of the squares of the difference in distances between the molecule and the reference algorithm is monotonically increasing, which may not be the case for more complicated algorithms. Using this method, many locations may be rejected without calculating the complete a sum of the squares of the difference in distances between the molecule and the reference for that location.

Pre-defined criteria for a match may be defined. For example, the sum of the squares of the difference in distances between the molecule and the reference cannot exceed a threshold value. This threshold value may be chosen based on prior knowledge, a desired level of fit, at random or in any other way. The threshold may be complex including parameters such as the length of the molecule, the length of the reference, the number of occurrences of the probe sequence in the molecule, the number of occurrences of the probe sequence in the reference, the rate of cross-hybridization, the rate of non-hybridization and many other parameters.

Unusually large distance may be used as an anchor. For example, if the molecule has a distance of 100 and such large distances are rare in the reference, only locations on the reference that include a distance of at least 100 may be evaluated. In this way, many reference locations do not need to be evaluated.

Unusually small distance may be used as an anchor. For example, if the molecule has a distance of 100 and such small distances are rare in the reference, only reference locations that include a distance of 100 or less may be evaluated. In this way, many reference locations do not need to be evaluated.

Thresholds on the largest and smallest distance may also be used (for example, the largest distance for a given location on the reference cannot be more than 20% larger than the largest distance on the molecule).

An example is encapsulated in the software code in Appendix 2. This may be used to design optimal experiments as well as to assess power and accuracy and to map molecules.

### Mapping Multiple Molecules to Form a Consensus Bar-Code Map for a Given Sample

The method extends naturally to mapping multiple molecules. Combining data from more than one molecule has a number of advantages including, but not limited to, multiple overlapping molecules may reduce the error, multiple overlapping molecules may increase accuracy, multiple molecules allow the interrogation of several different regions of an individual sample, and/or multiple overlapping molecules allow interrogation of longer segments of a sample.

Combining data from more than one molecule has further advantage that multiple overlapping molecules may be mapped against each other, without need for a reference. This de novo bar-coding is especially useful when a sample varies greatly from the available reference. The process is analogous to mapping a molecule to the reference, except that a second molecule is used in place of the reference. Further, one molecule may be a subset of the other, but this need not be the case. The molecules may overlap by any amount. The larger the overlap, the easier it will be to position the two molecules against one another in most cases.

Moreover, multiple molecules may allow the formation of a consensus bar-code map of a sample. This might be the entire genome or any subset of the genome, the extension of the reference, thereby adding information to what is known about the reference, and/or the detection of errors in the reference, thereby adding information to what is known about the reference

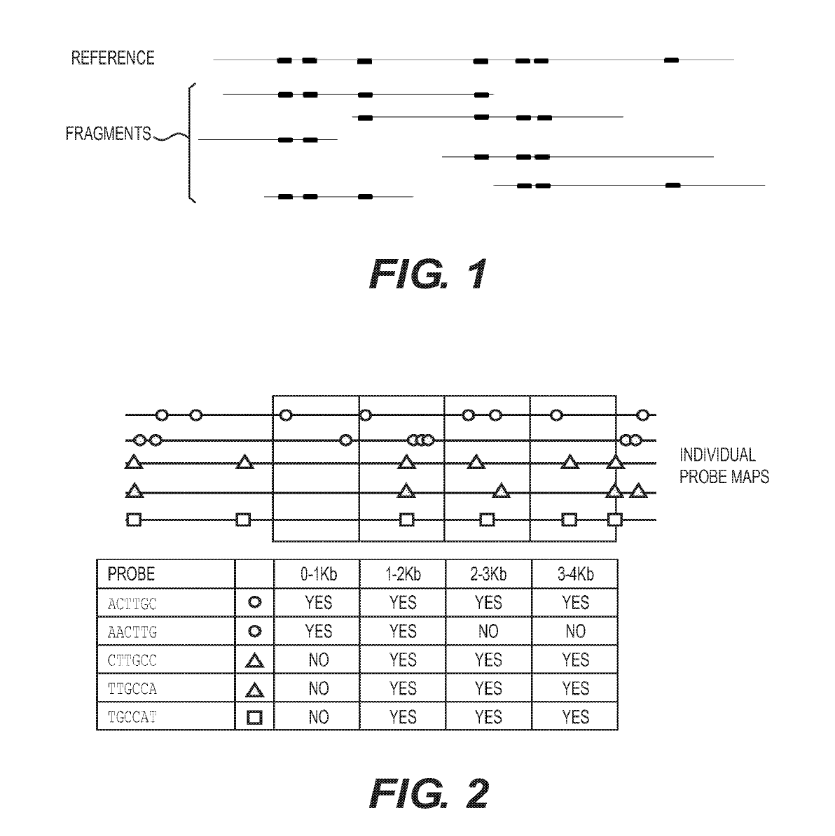

FIG. 1 shows the mapping of molecules either to a reference of to each other (de novo mapping).

Computer software for mapping molecules against a reference is given in Appendix 2. This software encapsulates a subset of the analyses described and is used for example purposes.

### Mapping Using Multiple Probes

The methods extend to the mapping of multiple different probes. For example, two separate 6 bp probes with different sequences may be used. They may be used in several different ways including, but not limited to, two or more probes may be labeled with different labels (for example, dyes that emit light at different wavelengths) and hybridized to the same molecule or set of molecules; two or more probes may be labeled with the same label and hybridized to the same molecule or set of molecules; two or more probes may be labeled with different labels (for example, different wavelength dyes) and hybridized to a different molecule or a different set of molecules; two or more probes may be labeled with the same label and hybridized to a different molecule or a different set of molecules; two or more probes may be hybridized in series wherein the first probe is hybridized, imaged and then removed before the second probe is hybridized and imaged with the process repeating for subsequent probes; and/or two or more probes may be hybridized in series. That is, the first probe is hybridized, imaged before the second probe is hybridized and imaged with the process repeating for subsequent probes.

An example is encapsulated in the software code in Appendix 2. This may be used to design optimal experiments as well as to assess power and accuracy and to map molecules.

### Integrating Multiple Probe Maps

Integrating bar-code maps from different probes has a number of advantages including, but not limited to, increasing the resolution of the integrated map compared to one or more of the individual maps, eliminating error by building a consensus from the individual consensus maps, improving accuracy by building a consensus from the individual consensus maps, and/or enabling sequencing by building a consensus from the individual consensus maps

Integration may be performed in a number of ways including, but not limited to, aligning some or all the individual probe maps to a reference, aligning some or all the individual probe maps against each other, and/or aligning some or all the individual probe maps against each other using a probe that is common to them all. For example, two probes would be used to build each consensus map—a universal probe and a map-specific probe. The universal probe would then be common to all the bar-code maps and be used to align them.

### Identifying Local Probe Sets

By stretching molecules and imaging them, locational information is retained that would be lost in a solution-based approach. Specifically, aligning multiple consensus bar-code maps for multiple probes allows the determination of which probes appear in a specific location or region. Several factors affect the ability to localize probes including, but not limited to, the accuracy of measurement of distance, the accuracy of alignment either against a reference or between the consensus bar-code maps, the number of probes used, the types of probes used, and/or the frequency of hybridization

FIG. 2 gives an example of assessing the presence of absence of five different probes whose consensus bar-code maps have been aligned. It assumes that the goal is to make lists of probes present in 1000 bp regions (which could, for example, be the resolution of the imaging). In the first 1000 bp region, only two of the five probes are observed (the ACTTGC probe shown in yellow and the AACTTG probe shown in green). Note, these two probes may be false positives caused by error (for example, cross-hybridization to related, but not identical sequences in the 1000 bp region). Similarly, the sequence of the three probes that are not observed may actually exist in the 1000 bp region and represent false negatives (for example, due to failure of hybridization). Algorithms for sequence assembly will ideally include methods for dealing with these potential false positive and false negative results.

### Sequencing by Hybridization

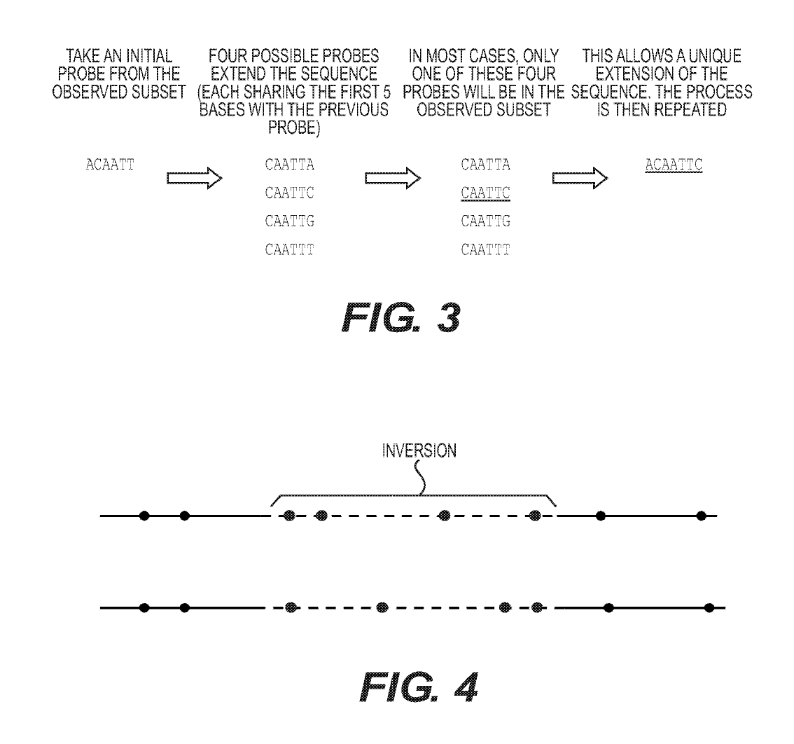

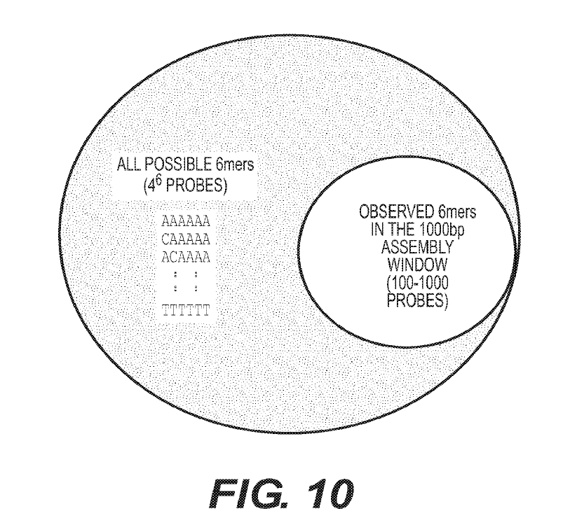

Hybridization is one of the most standard assays in molecular biology and has been applied to sequencing a number of times. However, Sequencing-by-Hybridization (SbH) has not been widely adopted, principally because it requires analysis of short fragments (usually PCR products) making it difficult to scale. Short fragments are required as they limit the number of probes observed. For example, with 6 base probes there are 4096 unique sequences. If the target is 6 bases long, only one of these will be present. If the target is the entire human genome, all 4096 will likely be observed as all 6 base sequences exist somewhere in the genome. This latter case is problematic, as if all the probes are present, it is impossible to know what order they occur along the genome. More useful is looking at a short fragment, say a 500 bp PCR product. In this case, at most 494 unique probes will be observed from the full set of 4096 (the idea is shown schematically in FIG. 10). This subset may then be ordered as shown in FIG. 3.

This approach has many advantages, not least that the assembly is very fast. However, it requires the genome to be fragmented into many small pieces and each of these to be interrogated separately. If the human genome is divided into non-overlapping 1 kb pieces, this would require approximately three million PCR reactions. Using locational information from stretched molecules alleviates this limitation as the resolution of the measurement of distance may be used in a manner analogous to a PCR product. That is, it is possible to identify the subset of probes that occur in a region of the genome. This is down by aligning the consensus bar-code maps for some or all of the probes and determining which probes lie in the region. No amplification or PCR is needed, so allowing the method to scale to entire genomes. As such local information revolutionizes the SbH assay if algorithms may be developed to construct and align the consensus bar-code maps. The method for constructing the sequence may include some or all of the following steps: determining distance estimates for each molecule for one or more probes; for each probe or set of probes, mapping the molecules either to a reference or to each other; for each probe or set of probes, constructing a consensus bar-code map; aligning the consensus bar-code maps; determining the subset of probes (which will be between none and all of them) that occur in a given region (that may be of arbitrary size); assembling the subset of probes for the given region using an algorithm; and/or repeating for overlapping regions (e.g. a sliding window approach) and build a consensus

Many factors may affect the exact steps in this process including, but not limited to, whether the molecule is single-stranded or double-stranded, the length of the molecules, the amount of stretching of the molecule, the distribution of stretching of the molecule, the length and type of probes, the number of probes, the completeness of the probe set (for example, for 6 bp oligos interrogating DNA, there are 4⁶=4096 possible probes, so data must be available from at least one and at most 4096 probes), the similarity of the probe sequences, the rate of cross-hybridization, the type of cross-hybridization (for example, GC-rich probes cross-hybridizing more than other probe types), the rate of missing probe data, the type of missing probe data (for example, palindromic probes such as ACGGCA failing more often than other types of probes), the resolution of the instrument used to measure distance, the variance on the estimate of distance, the bias in the measurement of distance, the accuracy of mapping individual molecules either to a reference or to each other, the accuracy of alignment of the consensus bar-code maps, the number of consensus bar-code maps, the use of a universal probe to align the consensus bar-code maps, the size of the region for which the subset of observed probes was calculated, the sequence of the region (for example, the method may work less well for repetitive sequences), the variance of the sample's sequence from the reference sequence, the specific differences between the sample's sequence from the reference sequence, the number of probes observed in the region, and/or the specific probes observed in the region. In some embodiments, both strands may be used to improve accuracy of assembly. Left-over or unused probes may be used to infer potential variants that may have been missed in the initial assembly

An example is encapsulated in the software code in Appendix 1. This may be used to design optimal experiments as well as to assess power and accuracy and to map molecules.

### Unused Probes

If a set of probes is observed in a given assembly window, the expectation would be that they are all used in the process of assembling the sequence. If some probes are not required for the assembly, it is possible something is wrong with the assembly. One possibility is that they are the result of cross-hybridization, imprecise localization or other types of error. Another is that there is a sequence, variant or element that is being missed in the assembly. For example, if the probes are related, they may define a particular sequence. As an example, suppose the set of observed probes that were not used in the assembly is {AAACT, AACTA, ACTAA, CTAAA, TAAAA}. A separate assembly may be performed on these probes. A maximum parsimony tiling algorithm would reconstruct a sequence AAACTAAAA, as this uses all the probes to build a consistent assembled sequence. There are a number or potential causes including, but not limited to, error in the location of the probe hybridization events, cross-hybridization, incorrect assembly, an inferior algorithm for assembly, a chance result, contamination with another sample, or another part of the target sample, an incorrect reference, and/or an genetic variant

Software code for identifying and interpreting these unused probes is included in Appendix 1. This software encapsulates a subset of the analyses described and is used for example purposes.

### Double-Stranded Analysis

Using double-stranded DNA presents a variety of issues including, but not limited to, the average spacing of between targets of the probes may be smaller compared to a single-stranded DNA, the number of probes hybridization events may be higher in a given assembly window, an different number of probes may be seen in a given assembly window than would be observed using single-stranded DNA, and/or assembly algorithms designed for single-stranded analysis may preform differently, less well or in other undesired ways.

Typically, more probes are observed in an assembly window for double-stranded DNA than for single-stranded DNA. This may cause a reduction in the power to correctly assemble or accuracy of the assembly as more potential assemblies may be possible with the larger set of probes, although this will depend on the specific algorithm. A way to deal with this is to assembly both DNA strands using the same probe set. In the simplest case this may be done independently. More complex algorithms may have additional features including, but not limited to, assemble both strand simultaneously, assemble one strand and then assemble the other strand, assemble one strand and then use the complement of this first strand as the reference for the other strand during assembly, assemble one strand and then assemble the second strand if there are unused probes in the observed probe set for the assembly region, and/or match the pairs of probes in the observed probe set for the assembly region (i.e. examine if the probe and its complement are both present).

Analyses show the benefits of single-stranded and double-stranded DNA. For former has fewer probes in a given assembly region, but lacks the ability to assemble both strands simultaneously. Quantification of these factors for a given experimental design or probe set will be critical in maximizing the accuracy of assembly.

An example is encapsulated in the software code in Appendix 1. This may be used to design optimal experiments as well as to assess power and accuracy and to map molecules.

### Missing Probes and Cross-Hybridization

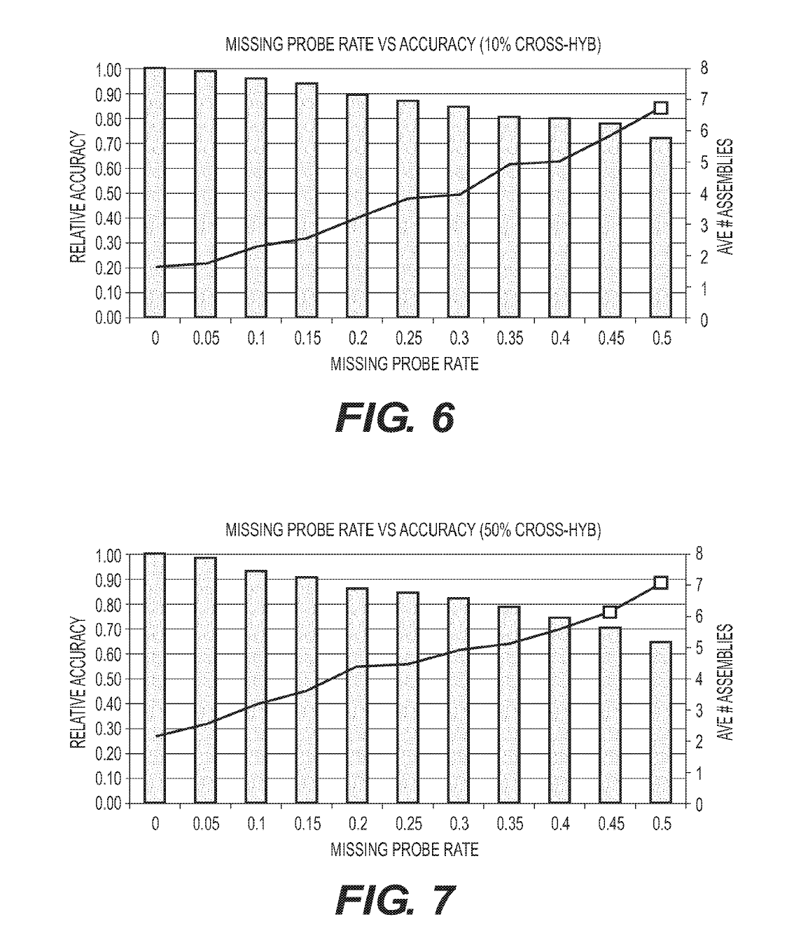

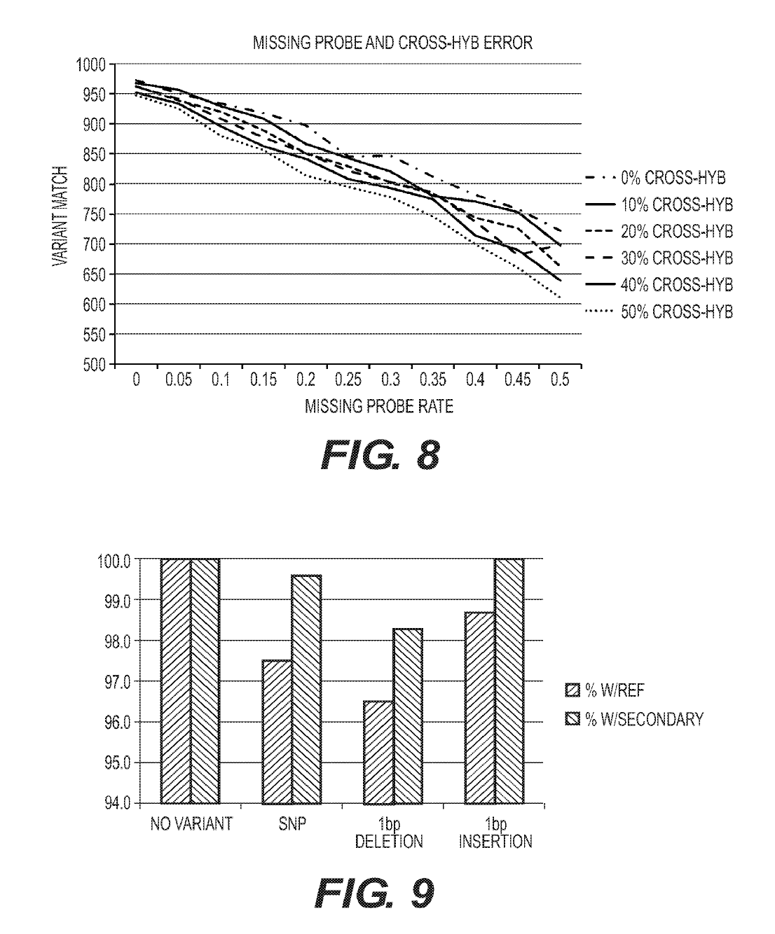

The effects of missing probes and cross-hybridization may play an important part in the design of the probe set and in the analysis of data in both structural variation detection and sequencing. FIGS. 6 through 8 show the role these factors play on the ability to correct assembly sequence. These analyses may be used in optimizing the experimental design.

An example is encapsulated in the software code in Appendix 1. This may be used to design optimal experiments as well as to assess power and accuracy and to map molecules.

### Structural Variation Detection

The consensus bar-code maps allow the rapid detection of structural variation between the sample and a reference (where the reference may be any other sample. For example, if could be a tumor-germline pair from a single cancer patient). FIG. 4 shows how a consensus bar-code map for a specific sample may be compared against a reference to identify an inversion. More complex algorithms may incorporate missing data, error, uncertainty, multiple samples, contamination and other factors.

Types of genetic variation that may be detected using these algorithms include, but are not limited to, inversions, deletions, amplifications, copy number change, translocations, reciprocal translocations, duplications, chimeras, complex rearrangements, and/or polysomy (for example, Trisomy).

### Case Study for Mapping Molecules to a Reference

Data was simulated for molecules of varying lengths, including 20,000 bp and 50,000 bp. The sequence of the molecules was taken from the human genome reference sequence as available in Wolfram's Mathematica package in 2011 (reference.wolfram.com/mathematica/ref/GenomeData.html).

A sum of squares of the difference in distance s between the molecule and the reference was used. Other measures of fit were also tested.

Error was introduces into the estimation of the distances for the molecules. It has a Gaussian (Normal) distribution with mean of 0 bp standard deviation of 1,000 bp. Other error functions were also tested.

Computer software was written in Mathematica to identify the location of the molecule against the reference sequence (Appendix 2).

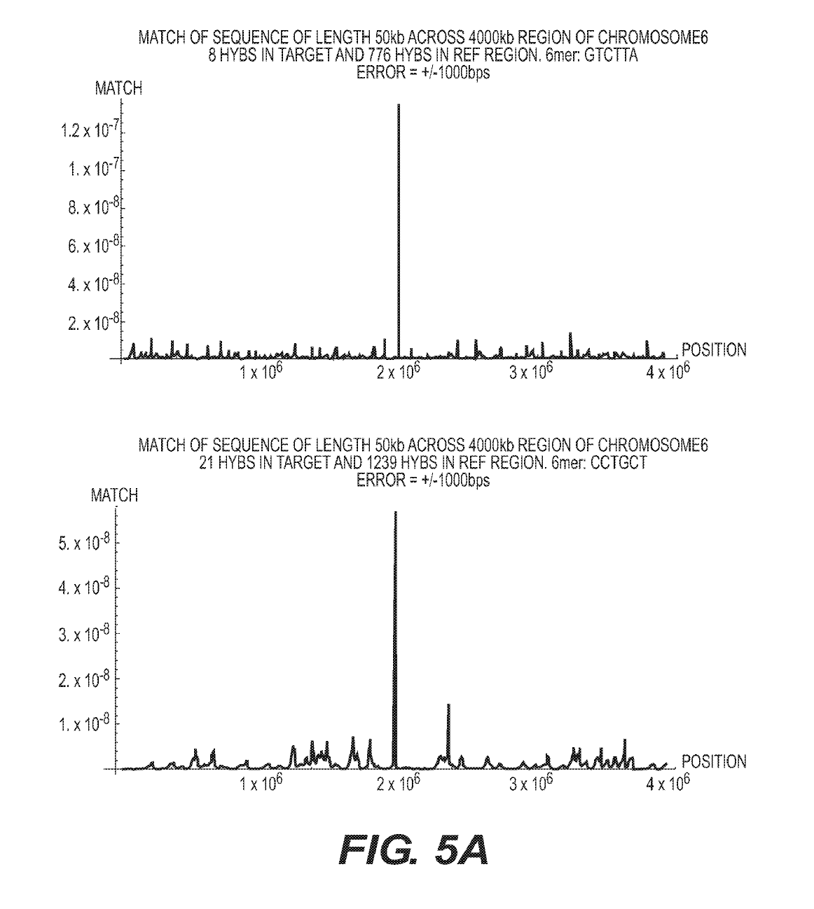

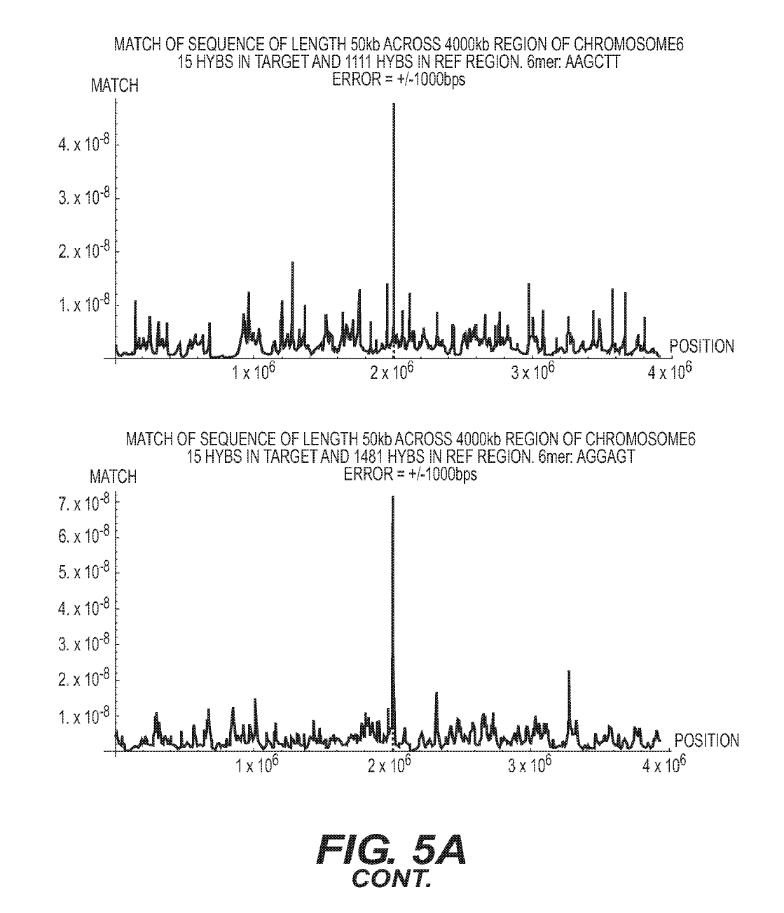

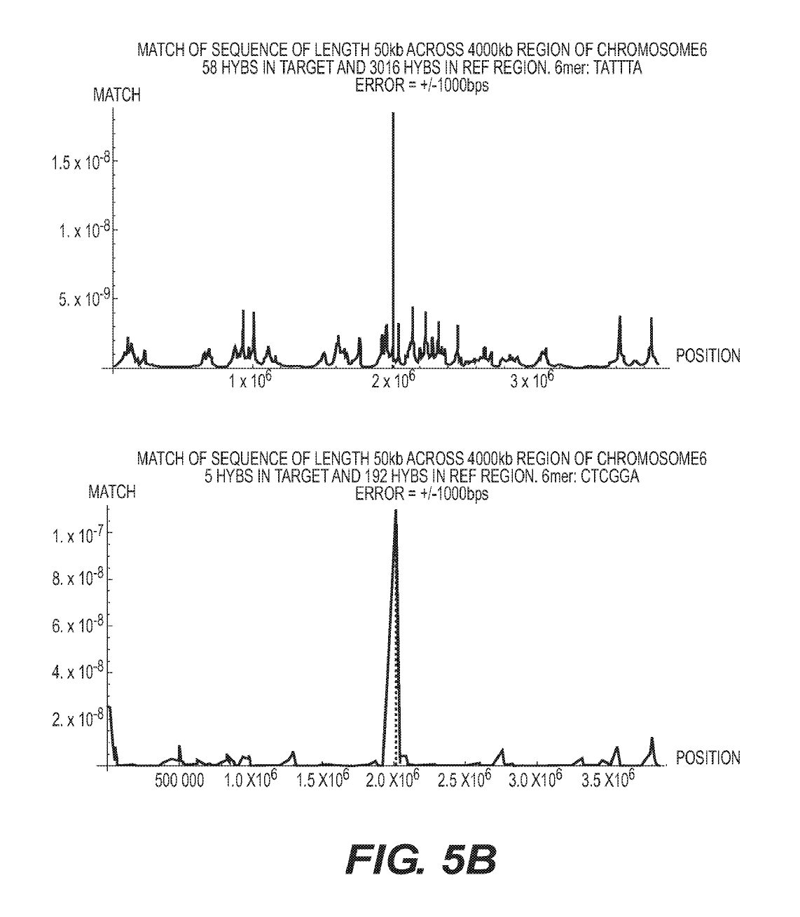

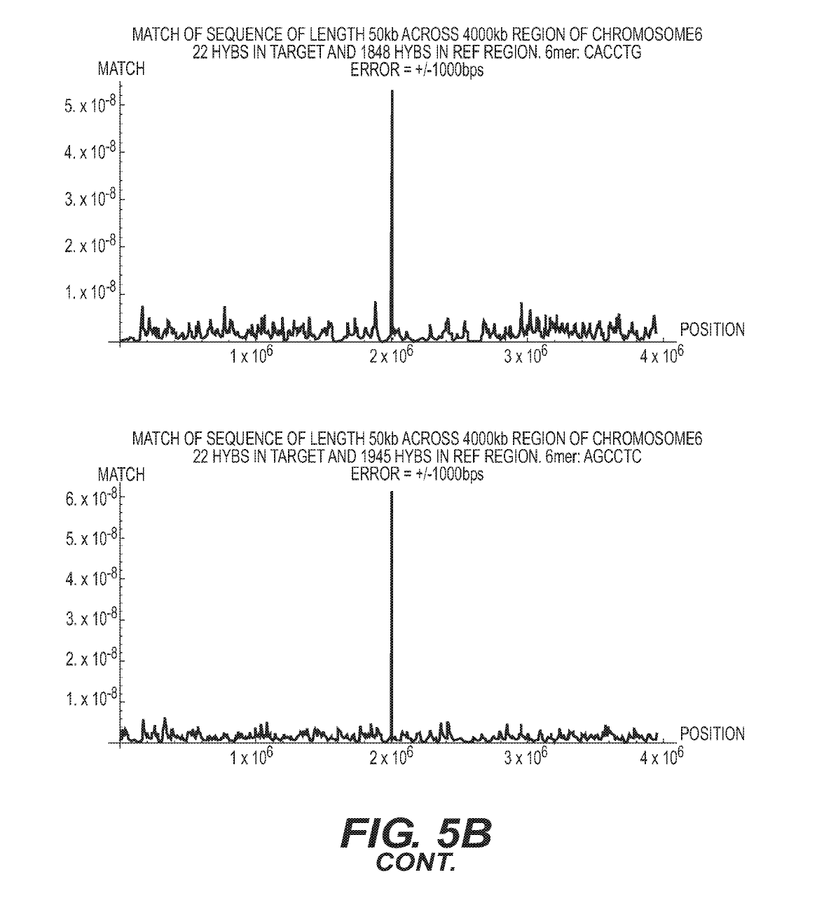

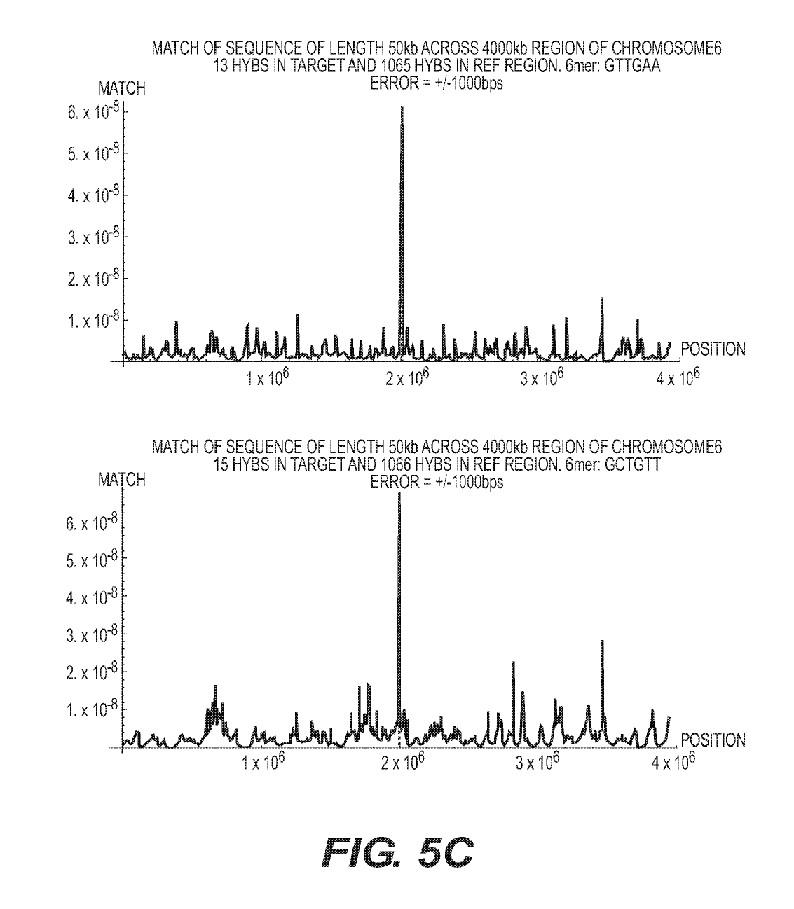

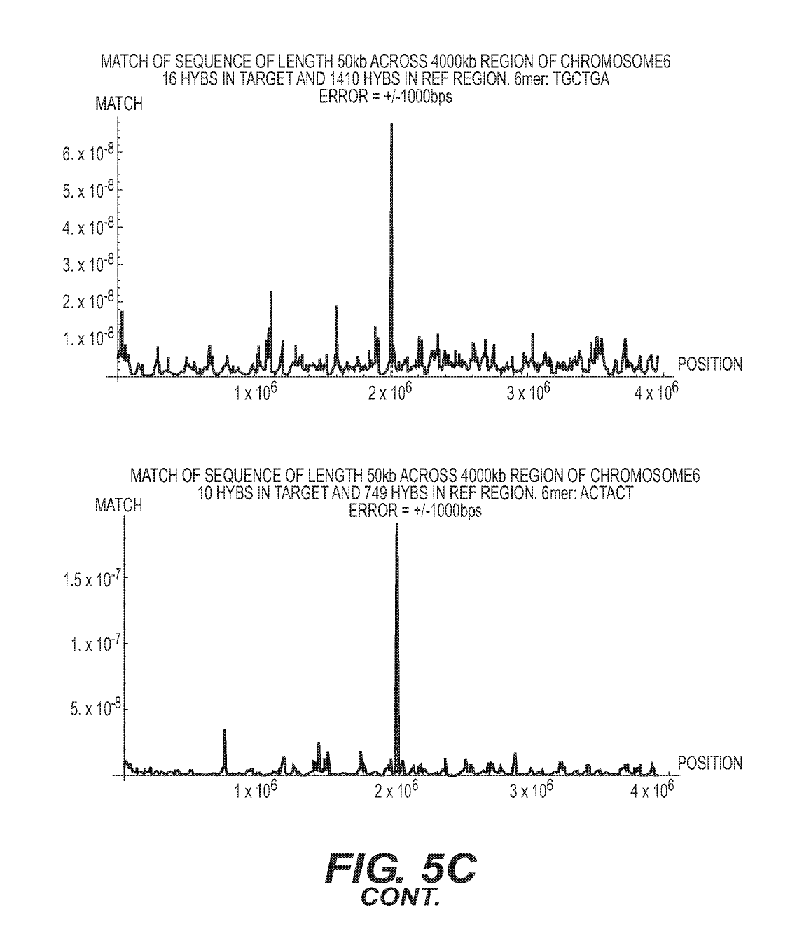

FIG. 5A, FIG. 5B, and FIG. 5C shows examples of the mapping of the molecules taken from human chromosome 6 to the region of chromosome 6 from which they were taken. In all cases, the correct position is at the center of each chart. Higher numbers represent a better match based on the comparison of the distance vectors.

### Case Study for Assembling Sequence Using Sequencing by Hybridization (SbH) on Stretched Molecules

Data was simulated for molecules of varying lengths, including 20,000 bp and 50,000 bp. The sequence of the molecules was taken from the human genome reference sequence as available in Wolfram's Mathematica package in 2011 (reference.wolfram.com/mathematica/ref/GenomeData.html).

Assembly windows of different size were tested including 500 bp, 800 bp, 1,000 bp, 1500 bp and 2000 bp.

A variety of errors were modeled including, but not limited to, cross-hybridization at various rates, cross-hybridization based on various sub-matches of the sequence, and/or missing probes at various rates

### Probe Optimization Example

Probes were optimized based on the ability to reconstruct a reference sequence taken from the human genome. Various 1000 bp segments of human chromosome 6 (the reference for these analyses) were examined and the set of probes of a specific type that are represented in the reference was identified. This set of probes was then used to re-construct the part or all of the reference. In a more complicated set of studies, a single-base change was introduced into the reference. The ability to identify this variant was then quantified for probes of different design. Table 1 shows results for some of the probe types tested. Parameters investigated included probe length, length of specific sequence, length of universal nucleotide sequence (i.e. sequence that matches any nucleotide), number of universal nucleotide sequence, and locations of universal nucleotide sequence. Many reference sequences were examined for each probe design. Importantly, these analyses show that the additional of universal nucleotides, spacers or gaps increases the ability to correctly assembly sequence. This fundamentally changes the design of probes in sequencing-by-hybridization experiments.

Example code written in Mathematic is given in Appendix 1.

### Cross-Hybridization

Probe designs were examined in the context of cross-hybridization. In the example, cross hybridization is measured as the probability that a probe hybridizes to a sequence that is not its perfect target. Cross-hybridization was modeled by assuming that a probe is more likely to hybridize to a related sequence than to a random sequence. In the example presented here, it was assumed that cross-hybridization occurred with a pre-defined probability at any position in the reference where the first 5 bp of the probe matched the target and the 6^{th} base could be any nucleotide that is not a match. So if A is a correct match and B is an in correct match, a probe cross-hybridized to the sequence AAAAAB with a predefined probability. For any given location where cross-hybridization could occur, the cross-hybridization was determined by generating a random number between 0 and 1 using Mathematica's inbuilt function and if this was less than the predefined cross-hybridization rate then a cross-hybridization event was assumed to have occurred.

In most cases, cross-hybridization was less deleterious to the ability to assembly sequence than missing probes. That is, 10% cross-hybridization reduced accuracy of assembly more than 10% missing probes. This has important ramifications for the design of the probe set. In this case, it would be better to optimize the hybridization conditions to increase the number of hybridization events, even if this leads to some cross-hybridization. Further, it will be often be better to include probes in the analysis, even if they have relatively high levels of cross-hybridization rather than exclude them from the analysis. These analyses enable the sequencing-by-hybridization assay, as they show that even imperfect probes may provide valuable data.

TABLE 1
Results for novel assembly algorithms that show optimization of a variety of parameters.
Category No.

| 1 | 2 | 3 | 4 | 5 | 6 | 7 | 8 | 9 | 10 | 11 | 12 | 13 | 14 |
|---|---|---|---|---|---|---|---|---|---|---|---|---|---|
| 5 | 0, 0, 3, 0, 0 | SNP | 200 | 0.2 | 0.75 | 38 | 962 | 935 | 38 | 1000 | 96.2 | 93.5 | 100 |
| 5 | 0, 0, 3, 0, 0 | SNP | 500 | 0.2 | 0.75 | 141 | 859 | 624 | 141 | 1000 | 85.9 | 62.4 | 100 |
| 5 | 0, 0, 3, 0, 0 | SNP | 800 | 0.2 | 0.75 | 350 | 650 | 160 | 350 | 1000 | 65 | 16 | 100 |
| 5 | 0, 0, 3, 0, 0 | SNP | 1000 | 0 | 0 | 343 | 657 | 148 | Not Tested | 1000 | 65.7 | 14.8 | |
| 5 | 1, 1, 1, 1, 0 | SNP | 1000 | 0 | 0 | 364 | 636 | 76 | Not Tested | 1000 | 63.6 | 7.6 | |
| 5 | 1, 1, 1, 1, 0 | SNP | 1000 | 0.2 | 0.75 | 439 | 561 | 36 | 439 | 1000 | 56.1 | 3.6 | 100 |
| 5 | 5, 5, 5, 5, 0 | SNP | 1000 | 0.2 | 0.75 | 269 | 731 | 162 | 192 | 1000 | 73.1 | 16.2 | 92.3 |
| 5 | 3, 3, 3, 3, 0 | SNP | 1000 | 0.2 | 0.75 | 253 | 747 | 176 | 148 | 1000 | 74.7 | 17.6 | 89.5 |
| 6 | 0, 0, 0, 0, 0 | SNP | 1000 | 0 | 0 | 64 | 936 | 789 | Not Tested | 1000 | 93.6 | 78.9 | |
| 6 | 0, 0, 20, 0, 0 | SNP | 1000 | 0.2 | 0.75 | 35 | 965 | 915 | 25 | 1000 | 96.5 | 91.5 | 99 |
| 6 | 0, 0, 3, 0, 0 | SNP | 200 | 0.2 | 0.75 | 25 | 975 | 970 | 25 | 1000 | 97.5 | 97 | 100 |
| 6 | 0, 0, 3, 0, 0 | SNP | 500 | 0.2 | 0.75 | 33 | 967 | 956 | 33 | 1000 | 96.7 | 95.6 | 100 |
| 6 | 0, 0, 3, 0, 0 | SNP | 800 | 0.2 | 0.75 | 42 | 958 | 931 | 42 | 1000 | 95.8 | 93.1 | 100 |
| 6 | 0, 0, 3, 0, 0 | SNP | 800 | 0.2 | 0.75 | 45 | 955 | 905 | 42 | 1000 | 95.5 | 90.5 | 99.7 |
| 6 | 0, 0, 3, 0, 0 | 1 bp | 1000 | 0 | 1 | 29 | 951 | 116 | 29 | 980 | 97.0 | 11.8 | 100 |
| | | Deletion | | | | | | | | | | | |
| 6 | 0, 0, 3, 0, 0 | 1 bp | 1000 | 0 | 1 | 43 | 925 | 7 | 43 | 968 | 95.6 | 0.7 | 100 |
| | | Insertion | | | | | | | | | | | |
| 6 | 0, 0, 3, 0, 0 | SNP | 1000 | 0 | 0 | 40 | 960 | 925 | Not Tested | 1000 | 96 | 92.5 | |
| 6 | 0, 0, 3, 0, 0 | SNP | 1000 | 0.05 | 0 | 44 | 956 | 922 | Not Tested | 1000 | 95.6 | 92.2 | |
| 6 | 0, 0, 3, 0, 0 | SNP | 1000 | 0.1 | 0 | 45 | 955 | 907 | Not Tested | 1000 | 95.5 | 90.7 | |
| 6 | 0, 0, 3, 0, 0 | SNP | 1000 | 0.2 | 0 | 48 | 952 | 896 | Not Tested | 1000 | 95.2 | 89.6 | |
| 6 | 0, 0, 3, 0, 0 | SNP | 1000 | 0.2 | 1 | 38 | 962 | 908 | 38 | 1000 | 96.2 | 90.8 | 100 |
| 6 | 0, 0, 3, 0, 0 | SNP | 1000 | 0.2 | 0.75 | 36 | 964 | 906 | 36 | 1000 | 96.4 | 90.6 | 100 |
| 6 | 0, 0, 3, 0, 0 | SNP | 1000 | 0.25 | 0 | 50 | 950 | 891 | Not Tested | 1000 | 95 | 89.1 | |
| 6 | 0, 0, 3, 0, 0 | SNP | 1000 | 0.3 | 0 | 53 | 947 | 880 | Not Tested | 1000 | 94.7 | 88 | |
| 6 | 0, 0, 3, 0, 0 | SNP | 1000 | 0.4 | 0 | 61 | 939 | 869 | Not Tested | 1000 | 93.9 | 86.9 | |
| 6 | 0, 0, 3, 0, 0 | SNP | 1000 | 0.5 | 0 | 61 | 939 | 838 | Not Tested | 1000 | 93.9 | 83.8 | |
| 6 | 0, 0, 3, 0, 0 | SNP | 1000 | 0.8 | 0.75 | 71 | 929 | 790 | 71 | 1000 | 92.9 | 79 | 100 |
| 6 | 0, 0, 3, 0, 0 | SNP | 1200 | 0.2 | 0.75 | 60 | 940 | 877 | 60 | 1000 | 94 | 87.7 | 100 |
| 6 | 0, 0, 3, 0, 0 | SNP | 1500 | 0.2 | 0.75 | 87 | 913 | 772 | 87 | 1000 | 91.3 | 77.2 | 100 |
| 6 | 0, 0, 3, 0, 0 | SNP | 1800 | 0.2 | 0.75 | 360 | 661 | 0 | 286 | 1021 | 64.7 | 0 | 92.8 |
| 6 | 0, 0, 3, 0, 0 | SNP | 2000 | 0.2 | 0.75 | 410 | 621 | 0 | 323 | 1031 | 60.2 | 0 | 91.6 |
| 6 | 0, 0, 6, 0, 0 | SNP | 1000 | 0 | 0 | 39 | 961 | 927 | Not Tested | 1000 | 96.1 | 92.7 | |
| 6 | 0, 0, 6, 0, 0 | SNP | 1000 | 0.2 | 0.75 | 50 | 950 | 875 | 50 | 1000 | 95 | 87.5 | 100 |
| 6 | 0, 10, 10, 10, 0 | SNP | 1000 | 0.2 | 0.75 | 31 | 969 | 945 | 19 | 1000 | 96.9 | 94.5 | 98.8 |
| 6 | 0, 20, 0, 20, 0 | SNP | 1000 | 0.2 | 0.75 | 29 | 971 | 932 | 19 | 1000 | 97.1 | 93.2 | 99 |
| 6 | 0, 3, 0, 3, 0 | SNP | 1000 | 0.2 | 0.75 | 52 | 948 | 903 | 51 | 1000 | 94.8 | 90.3 | 99.9 |
| 6 | 0, 3, 3, 0, 0 | SNP | 1000 | 0.2 | 0.75 | 41 | 959 | 931 | 39 | 1000 | 95.9 | 93.1 | 99.8 |
| 6 | 0, 3, 3, 3, 0 | SNP | 500 | 0.2 | 0.75 | 22 | 978 | 972 | 21 | 1000 | 97.8 | 97.2 | 99.9 |
| 6 | 0, 3, 3, 3, 0 | SNP | 1000 | 0.2 | 0.75 | 40 | 960 | 939 | 39 | 1000 | 96 | 93.9 | 99.9 |
| 6 | 0, 0, 3, 0, 0 | SNP | 1000 | 0.2 | 0 | 48 | 952 | 896 | Not Tested | 1000 | 95.2 | 89.6 | |
| 6 | 0, 0, 3, 0, 0 | SNP | 1000 | 0.2 | 1 | 38 | 962 | 908 | 38 | 1000 | 96.2 | 90.8 | 100 |
| 6 | 0, 0, 3, 0, 0 | SNP | 1000 | 0.2 | 0.75 | 36 | 964 | 906 | 36 | 1000 | 96.4 | 90.6 | 100 |
| 6 | 0, 0, 3, 0, 0 | SNP | 1000 | 0.25 | 0 | 50 | 950 | 891 | Not Tested | 1000 | 95 | 89.1 | |
| 6 | 0, 40, 0, 40, 0 | SNP | 1000 | 0.2 | 0.75 | 75 | 925 | 766 | 65 | 1000 | 92.5 | 76.6 | 99 |
| 6 | 0, 5, 20, 5, 0 | SNP | 1000 | 0.2 | 0.75 | 25 | 975 | 939 | 15 | 1000 | 97.5 | 93.9 | 99.0 |
| 6 | 0, 5, 40, 5, 0 | SNP | 1000 | 0.2 | 0.75 | 48 | 952 | 841 | 39 | 1000 | 95.2 | 84.1 | 99.1 |
| 6 | 0, 5, 5, 5, 0 | 1 bp | 300 | 0.2 | 0.75 | 17 | 978 | 203 | 17 | 995 | 98.3 | 20.4 | 100.0 |
| | | Insertion | | | | | | | | | | | |
| 6 | 0, 5, 5, 5, 0 | 1 bp | 300 | 0.2 | 0.75 | 16 | 979 | 414 | 16 | 995 | 98.4 | 41.6 | 100.0 |
| | | Deletion | | | | | | | | | | | |
| 6 | 0, 5, 5, 5, 0 | SNP | 300 | 0.2 | 0.75 | 25 | 975 | 968 | 23 | 1000 | 97.5 | 96.8 | 99.8 |
| 6 | 0, 5, 5, 5, 0 | 1 bp | 500 | 0.2 | 0.75 | 18 | 980 | 489 | 17 | 998 | 98.2 | 49.0 | 99.9 |
| | | Deletion | | | | | | | | | | | |
| 6 | 0, 5, 5, 5, 0 | 1 bp | 500 | 0.2 | 0.75 | 17 | 980 | 769 | 16 | 997 | 98.3 | 77.1 | 99.9 |
| | | Insertion | | | | | | | | | | | |
| 6 | 0, 5, 5, 5, 0 | SNP | 500 | 0.2 | 0.75 | 19 | 981 | 974 | 16 | 1000 | 98.1 | 97.4 | 99.7 |
| 6 | 0, 5, 5, 5, 0 | 1 bp | 750 | 0.2 | 0.75 | 21 | 979 | 219 | 20 | 1000 | 97.9 | 21.9 | 99.9 |
| | | Deletion | | | | | | | | | | | |
| 6 | 0, 5, 5, 5, 0 | SNP | 750 | 0.2 | 0.75 | 25 | 975 | 961 | 19 | 1000 | 97.5 | 96.1 | 99.4 |
| 6 | 0, 5, 5, 5, 0 | 1 bp | 750 | 0.2 | 0.75 | 20 | 979 | 138 | 20 | 999 | 98.0 | 13.8 | 100.0 |
| | | Insertion | | | | | | | | | | | |
| 6 | 0, 5, 5, 5, 0 | No | 1000 | 0.2 | 0.75 | 1000 | 1000 | 1000 | 0 | 1000 | 100.0 | 100.0 | 100.0 |
| | | Variant | | | | | | | | | | | |
| 6 | 0, 5, 5, 5, 0 | SNP | 1000 | 0.2 | 0.75 | 25 | 975 | 950 | 21 | 1000 | 97.5 | 95 | 99.6 |
| 6 | 0, 5, 5, 5, 0 | 1 bp | 1000 | 0.2 | 0.75 | 35 | 962 | 0 | 18 | 997 | 96.5 | 0.0 | 98.3 |
| | | Deletion | | | | | | | | | | | |
| 6 | 0, 5, 5, 5, 0 | 1 bp | 1000 | 0.2 | 0.75 | 13 | 985 | 4 | 13 | 998 | 98.7 | 0.4 | 100.0 |
| | | Insertion | | | | | | | | | | | |
| 6 | 0, 7, 7, 7, 0 | SNP | 1000 | 0.2 | 0.75 | 29 | 971 | 938 | 17 | 1000 | 97.1 | 93.8 | 98.8 |
| 6 | 1, 1, 1, 1, 1 | SNP | 1000 | 0 | 0 | 39 | 961 | 847 | Not Tested | 1000 | 96.1 | 84.7 | |
| 6 | 1, 1, 1, 1, 1 | SNP | 1000 | 0.2 | 0 | 57 | 943 | 793 | Not Tested | 1000 | 94.3 | 79.3 | |
| 6 | 10, 10, 10, 10, 10 | SNP | 1000 | 0.2 | 0.75 | 41 | 959 | 826 | 31 | 1000 | 95.9 | 82.6 | 99 |
| 6 | 20, 20, 20, 20 | SNP | 1000 | 0.2 | 0.75 | 38 | 962 | 816 | 29 | 1000 | 96.2 | 81.6 | 99.1 |
| 6 | 3, 0, 0, 0, 3 | SNP | 1000 | 0.2 | 0.75 | 42 | 958 | 927 | 38 | 1000 | 95.8 | 92.7 | 99.6 |
| 6 | 3, 0, 3, 0, 3 | SNP | 1000 | 0.2 | 0.75 | 39 | 961 | 922 | 37 | 1000 | 96.1 | 92.2 | 99.8 |
| 6 | 3, 3, 3, 3, 3 | SNP | 1000 | 0 | 0 | 45 | 955 | 861 | Not Tested | 1000 | 95.5 | 86.1 | |
| 6 | 3, 3, 3, 3, 3 | SNP | 1000 | 0.2 | 0.75 | 63 | 937 | 773 | 59 | 1000 | 93.7 | 77.3 | 99.6 |
| 6 | 5, 5, 5, 5, 5 | SNP | 1000 | 0.2 | 0.75 | 55 | 945 | 816 | 42 | 1000 | 94.5 | 81.6 | 98.7 |
| 6 | 6, 6, 6, 6, 6 | SNP | 1000 | 0 | 0 | 49 | 951 | 861 | Not Tested | 1000 | 95.1 | 86.1 | |

TABLE 2
Column Heading Descriptions for Table 1

| Column | Description |
|---|---|
| 1. Nmer | Number of specific nucleotides in each probe |
| 2. Spacing | The position of universal nucleotides (or gaps or |
| | spacers) in the probe. For example, if the probe is |
| | has 6 specific bases ACTGAC and the spacing |
| | vector is {0, 3, 0, 3, 0} then the probe is |
| | ACNNNTGNNNAC where N represents the |
| | universal nucleotides (or gaps or spacers). That is, |
| | the spacing vector has entries for the spacing |
| | between each consecutive specific nucleotide. As |
| | such, the length of the spacing vector is one less than |
| | the number of specific nucleotides. A spacing vector |
| | {0, 0, 0, 0, 0} would need the probe is in its original |
| | form ACTGAC. The sum of the entries in the |
| | spacing vector gives the total number of universal |
| | nucleotides (or gaps or spacers). The sum of the |
| | entries in the spacing vector plus Nmer gives the |
| | total length of the probe. |
| 3. Variant | The type of de novo variant introduced into the |
| | reference (SNP = Single Nucleotide Polymorphism) |
| 4. Assembly Window Size | The size of the segment to be assembled |
| 5. Cross-Hybridization | The probability of cross-hybridization |
| 6. Secondary Match | The proportion of the probes need to define the |
| | variant (6 for a 1bp change) that are present in the |
| | set of unused probes |
| 7. Consensus Match | The number of times the reference is an equal or |
| | better match than the true variant sequence |
| 8. Correct (Var March when variant is present) | The number of times the variant was correctly |
| | identified (that is, the correct nucleotide change at |
| | the correct location) |
| 9. Correct & Unique | The number of times the variant was correctly |
| | identified (that is, the correct nucleotide change at |
| | the correct location) and this was a better match than |
| | any other assembly tested for the given algorithm |
| 10. Secondary Identification where Ref is True | When the reference has the same or better match |
| | than any other sequence, a test may be performed |
| | using unused probes (see text above). This provides |
| | another way of detecting variants. This column |
| | gives the number of times a variant was detected in |
| | this secondary analysis |
| 11. Total | The total number of regions or the Assembly |
| | Window Size that were assembled |
| 12. % w/Ref | The percent of times the assembled sequence was |
| | correct (including identifying the Variant) |
| 13. % unambiguous | The percent of times the correct sequence was |
| | unambiguously the best match. That is, no other |
| | tested assembly had an equal or better match. |
| 14. % w/Secondary | The percent of times the assembled sequence was |
| | correct (including identifying the Variant) either |
| | with primary analysis or with the secondary analysis |

1. A method of analyzing a nucleic acid sample, comprising selecting a group of one or more labeled oligonucleotide probe(s), contacting at least one of the group of the labeled oligonucleotide probe(s) to at least one nucleic acid molecule(s) from the nucleic acid sample, wherein the nucleic acid molecule(s) is stretched, and correlating one or more point(s) of contact to a structural characteristic of the nucleic acid sample.

2. The method according to claim 1, wherein the nucleic acid molecule(s) is deoxyribonucleic acid (DNA).

3. The method according to claim 1, wherein the method of contacting is hybridization or ligation.

4. The method according to claim 1, further comprising imaging points of contact along the nucleic acid molecules and measuring the distance between them.

5. The method according to claim 1, further comprising sequencing at least one part of the nucleic acid molecules using information on the points of contact and the distance between them.

6. The method according to claim 1, further comprising sequencing at least one part of the nucleic acid molecule(s), wherein the labeled oligonucleotide probe(s) are selected from a group of 4096 possible oligonucleotide probes having at least 6 nucleotides.

7. The method according to claim 6, wherein the labeled oligonucleotide probe(s) consists of a group of 4096 possible oligonucleotide probes having at least 6 nucleotides.

8. The method according to claim 7, wherein the nucleic acid molecule(s) is a whole genome sequence.

9. The method according to claim 1, further comprising detecting an error(s) in either the location of the contacting or the distance between contact points.

10. The method according to claim 1, further comprising detecting an error(s) in either the location of the contacting or the distance between contact points, and quantifying the error(s).

11. The method according to claim 1, further comprising detecting an error(s) in either the location of the contacting or the distance between contact points, and correcting the error(s).

12. The method according to claim 1, further comprising sequencing the nucleic acid molecule(s), reconstructing a nucleic acid sequence from the labeled oligonucleotide probe(s) that have not been contacted to the nucleic acid molecule(s), comparing the sequenced nucleic acid molecule(s) and the reconstructed nucleic acid sequence, and using this information in correcting an error(s).

13. The method according to claim 1, where the nucleic acid sample comprises either single or double stranded nucleic acid molecule(s), or a combination thereof.

14. The method according to claim 1, wherein the nucleic acid sample comprises double stranded nucleic acid molecules, and each step of the method is performed independently on each strand of nucleic acid molecule.

15. The method according to claim 1, wherein the labeled oligonucleotide probe(s) comprises a spacer.

16. The method according to claim 1, wherein the labeled oligonucleotide probe(s) comprises a spacer that is located to optimize reconstruction of genomic information.

17. The method according to claim 1, wherein the labeled oligonucleotide probe(s) comprises a spacer and/or a degenerative nucleotide, and the labeled oligonucleotide probe(s) comprises 6 or fewer non-spacer nucleotides.

18. The method according to claim 1, wherein the labeled oligonucleotide probe(s) is less than 30 nucleotide long.

19. The method according to claim 1, wherein the labeled oligonucleotide probe(s) is less than 10 nucleotide long.

20. The method according to claim 1, wherein the labeled oligonucleotide probe(s) is 6 nucleotide long.

21. The method according to claim 1, wherein the nucleic acid molecule is stretched before the contacting with the labeled oligonucleotide probe(s).

22. The method according to claim 1, wherein the nucleic acid molecule is stretched after the contacting by the labeled oligonucleotide probe(s).

23. The method according to claim 1, wherein the nucleic acid molecule(s) is not nicked by the labeled oligonucleotide probe(s).
